(19) United States
(12) Patent Application Publication
(10) Pub. No.: US 2024/0402332 A1
(43) Pub. Date: Dec. 5, 2024

(54) Device And Method For Estimating Radar Invisible Region Of Reinforced Concrete Structure

(71) Applicant: KEYTEC, Inc., Tokyo (JP)

(72) Inventor: Kazuhiko IWATA, Tokyo (JP)

(21) Appl. No.: 18/656,758

(22) Filed: May 7, 2024

(30) Foreign Application Priority Data
May 31, 2023 (JP) 2023-089996

(51) Int. Cl. G01S 13/88; G01N 33/38; G01S 13/06

(52) U.S. Cl. CPC G01S 13/88; G01N 33/383; G01S 13/06

(57) ABSTRACT

A device and a method for estimating a radar invisible region of a reinforced concrete structure are provided. The device has: a radar exploration result acquisition unit for a reinforced concrete structure; a vertex invisible waveform part information acquisition unit which acquires waveform information of a waveform part of a hyperbola waveform in which a vertex is invisible from the acquired radar exploration result; a vertex estimation means holding unit which holds vertex estimation means for estimating a vertex position of the vertex invisible waveform part on the basis of the vertex invisible waveform part information; and a vertex estimation unit which estimates a vertex position of the vertex invisible waveform part on the basis of the vertex invisible waveform part information and the vertex estimation means.

### CROSS REFERENCE TO RELATED APPLICATIONS

This application claims priority to Japanese Patent Application No. 2023-08996, filed May 31, 2023, which is expressly incorporated by reference herein in its entirety.

### TECHNICAL FIELD

The present invention relates to a device and a method for estimating a radar invisible region of a reinforced concrete structure, which can calculate positions of reinforcing bars disposed inside the reinforced concrete structure and estimate partially unknown position information of the reinforcing bars. It is to be noted that the reinforced bars of the present invention include both a reinforcing bar with a circular section called a round bar and a reinforcing bar with uneven surfaces called a deformed reinforcing bar, but in this Specification, for convenience, they are treated as the round bars each having a circular section.

### BACKGROUND ART

In recent years, in preparation for a large earthquake expected to occur in the near future and as countermeasures against aging, many reinforced concrete structures such as buildings and bridges have been reinforced. For this reinforcement, it is necessary to know the positions of reinforcing bars buried in the concrete of the reinforced concrete structures.

Conventionally, as a method of measuring the position of reinforcing bars that is employed when information on the reinforcing bars cannot be acquired from construction drawings and the like, a measuring method using an electromagnetic wave radar is known, for example. In this measuring method using the electromagnetic wave radar, a floor which is a reinforced concrete structure with reinforcing bars inside is scanned by the radar, a hyperbola waveform, which is information of a reflected wave which is reflected back from the reinforcing bar, is acquired during this radar scanning, and the position and depth of the reinforcing bars are measured from a position of a vertex thereof.

As shown in a two-dimensional image in FIG. 1A (an image of a radar screen displaying the time until receiving the reflected wave with respect to a position of the electromagnetic wave radar from the reinforcing bar), This hyperbola waveform is a symmetrical mountain-shaped waveform displayed with a reinforcing bar 0130 as a vertex, the reinforcing bar 0130 being measured on the basis of the time until receiving the reflected wave at a point of time when an electromagnetic wave radar 0120 passes immediately above and in a direction orthogonal to the reinforcing bar 0130 buried inside a floor 0100, which is a reinforced concrete structure. It is to be noted that the hyperbola waveform (information) will be described in detail in the following.

Examples of methods similar to this method of measuring the position of the reinforcing bars include an estimation method described in Japanese Patent No. 7043663.

However, in the conventional method of measuring the position of a reinforcing bars described above, if the floor 0100, which is a reinforced concrete structure, is blocked by a wall W as shown in FIG. 1B, the movement of the electromagnetic wave radar 0120 is restricted near the wall. Thus, with respect to the reinforcing bar 0130w buried in the floor 0100 in the vicinity of the wall W, even if a hyperbola waveform H related to the reinforcing bar 0130w in the vicinity of this wall W is acquired by performing radar scanning, the hyperbola waveform H would be a waveform in which only a skirt r (solid-line part) is visible but a vertex p (broken-line part) is invisible as can be seen from the comparison with the hyperbola waveform H related to the reinforcing bar 0130 buried at a position away from the wall W of the floor 0100. As a result, the position of the reinforcing bar 0130w located in the floor 0100 in the vicinity of the wall W cannot be measured, which is a problem, and solution to this problem has been a conventional problem.

The present invention was made in order to solve the aforementioned conventional problem and has an object to provide a device and a method for estimating a radar invisible region of a reinforced concrete structure, the device and method being capable of estimating a position of a reinforcing bar in the vicinity of a wall when a hyperbola waveform is acquired by radar scanning on a floor, which is a reinforced concrete structure, even if the hyperbola waveform related to the reinforcing bar buried in the floor in the vicinity of the wall is a waveform in which only a skirt part thereof is visible but a vertex is invisible.

### SUMMARY

In order to solve the aforementioned problem, the present invention provides a device and a method for estimating a radar invisible region of a reinforced concrete structure described below.

A first aspect of the present invention is configured to have: a radar exploration result acquisition unit for the reinforced concrete structure; a vertex invisible waveform part information acquisition unit which acquires waveform information of a waveform part of a hyperbola waveform in which a vertex is invisible from the acquired radar exploration result; a vertex estimation means holding unit which holds vertex estimation means for estimating a vertex position of the vertex invisible waveform part on the basis of the vertex invisible waveform part information; and a vertex estimation unit which estimates a vertex position of the vertex invisible waveform part on the basis of the vertex invisible waveform part information and the vertex estimation means.

Moreover, a second aspect of the present invention is configured such that: the device has a vertex visible waveform information acquisition unit which acquires waveform information of a vertex visible waveform; the vertex estimation means of the vertex estimation means holding unit is further configured to estimate the vertex position of the vertex invisible waveform part also on the basis of the vertex visible waveform information; and the vertex estimation unit estimates the vertex position of the vertex invisible waveform part on the basis of the vertex visible waveform information, the vertex invisible waveform part information, and the vertex estimation means.

Moreover, a third aspect of the present invention is configured to further have a vertex position information output unit which outputs the estimated vertex position information.

In addition, a fourth aspect of the present invention is configured such that the vertex estimation means is plural estimation means which estimates a shape of a hyperbola curve of the vertex invisible waveform part on the basis of a plurality of pieces of the vertex visible waveform information.

In addition, a fifth aspect of the present invention is configured such that the vertex estimation means includes specific dielectric constant estimation means which estimates the shape of the hyperbola curve of the vertex invisible waveform part by calculating a specific dielectric constant of a concrete on the basis of the vertex visible waveform information.

In addition, a sixth aspect of the present invention is configured such that the vertex invisible waveform part is a vertex invisible waveform part which is present in a difficult part where a radar exploration by a radar exploration device is difficult due to a shape of the concrete structure.

On the other hand, a method of operating a device according to a seventh aspect of the present invention, which is a computer, for estimating a radar invisible region of a reinforced concrete structure is configured to have: a radar exploration result acquisition step for a reinforced concrete structure; a vertex invisible waveform part information acquisition step for acquiring waveform information of a waveform part of a hyperbola waveform in which a vertex is invisible in the acquired radar exploration result; a vertex estimation means holding step for holding the vertex estimation means for estimating a vertex position of the vertex invisible waveform part on the basis of the vertex invisible waveform part information; and a vertex estimation step for estimating the vertex position of the vertex invisible waveform part on the basis of the vertex invisible waveform part information and the vertex estimation means.

Moreover, an eighth aspect of the present invention is configured such that: the method has a vertex visible waveform information acquisition step for acquiring waveform information of a vertex visible waveform; the vertex estimation means of the vertex estimation means holding step is further configured to estimate the vertex position of the vertex invisible waveform part also on the basis of the vertex visible waveform information; and the vertex estimation step estimates the vertex position of the vertex invisible waveform part on the basis of the vertex visible waveform information, the vertex invisible waveform part information, and the vertex estimation means.

In addition, a ninth aspect of the present invention is further configured to have a vertex position information output step for outputting the estimated vertex position information.

In addition, a tenth aspect of the present invention is configured such that the vertex estimation means is plural estimation means which estimates a shape of a hyperbola curve of the vertex invisible waveform part on the basis of a plurality of pieces of the vertex visible waveform information.

In addition, an eleventh aspect of the present invention is configured such that the vertex estimation means includes specific dielectric constant estimation means which estimates the shape of the hyperbola curve of the vertex invisible waveform part by calculating a specific dielectric constant of a concrete on the basis of the vertex visible waveform information.

In addition, a twelfth aspect of the present invention is configured such that the vertex invisible waveform part is a vertex invisible waveform part which is present in a difficult part where a radar exploration by a radar exploration device is difficult due to a shape of the concrete structure.

A method of estimating a radar invisible region of a reinforced concrete structure according to a thirteenth aspect of the present invention, which can be read and executed by a computer, is configured to have: a radar exploration result acquisition program of a reinforced concrete structure; a vertex invisible waveform part information acquisition program for acquiring waveform information of a waveform part of a hyperbola waveform in which a vertex is invisible in the acquired radar exploration result; a vertex estimation means holding program for holding the vertex estimation means for estimating a vertex position of the vertex invisible waveform part on the basis of the vertex invisible waveform part information; and a vertex estimation program for estimating the vertex position of the vertex invisible waveform part on the basis of the vertex invisible waveform part information and the vertex estimation means.

Moreover, a fourteenth aspect of the present invention is configured such that: the method has a vertex visible waveform information acquisition program for acquiring waveform information of a vertex visible waveform; the vertex estimation means of the vertex estimation means holding program is further configured to estimate the vertex position of the vertex invisible waveform part also on the basis of the vertex visible waveform information; and the vertex estimation program estimates the vertex position of the vertex invisible waveform part on the basis of the vertex visible waveform information, the vertex invisible waveform part information, and the vertex estimation means.

In addition, a fifteenth aspect of the present invention is further configured to have a vertex position information output program for outputting the estimated vertex position information.

In addition, a sixteenth aspect of the present invention is configured such that the vertex estimation means is plural estimation means which estimates a shape of a hyperbola curve of the vertex invisible waveform part on the basis of a plurality of pieces of the vertex visible waveform information.

In addition, a seventeenth aspect of the present invention is configured such that the vertex estimation means includes specific dielectric constant estimation means which estimates the shape of the hyperbola curve of the vertex invisible waveform part by calculating a specific dielectric constant of a concrete on the basis of the vertex visible waveform information.

In addition, an eighteenth aspect of the present invention is configured such that the vertex invisible waveform part is a vertex invisible waveform part which is present in a difficult part where a radar exploration by a radar exploration device is difficult due to a shape of the concrete structure.

The present invention provides an extremely excellent effect that, for example, when a hyperbola waveform is acquired by performing the radar scanning on a floor, a wall, a pillar, a ceiling, or the like, which is a reinforced concrete structure, the positions of reinforcing bars in the vicinity of the wall can be estimated even if the hyperbola waveform related to the reinforcing bars buried in the floor or the like in the vicinity of the wall is a waveform in which only a skirt part thereof is visible but the vertex is invisible.

### BRIEF DESCRIPTION OF THE DRAWINGS

FIG. 1A is a two-dimensional image schematically illustrating a relationship between a reinforcing bar and a hyperbola waveform when radar scanning is performed on a floor which is a reinforced concrete structure;

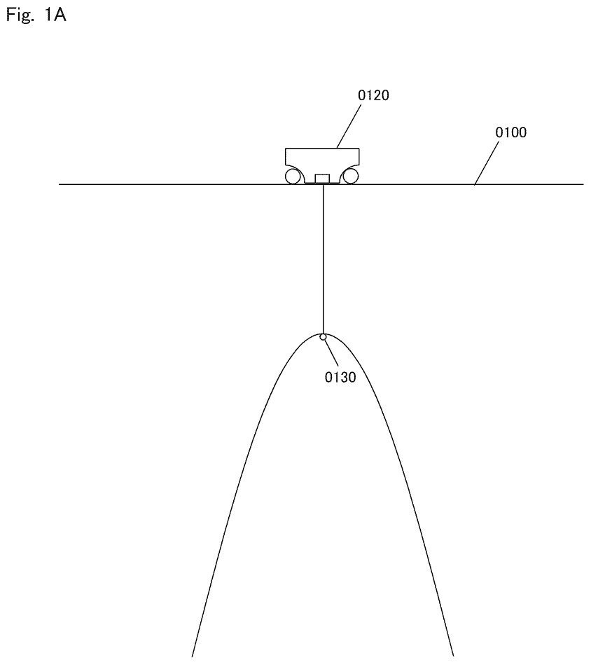

FIG. 1B is a two-dimensional image schematically illustrating a hyperbola waveform related to a reinforcing bar buried in the vicinity of a wall acquired by performing radar scanning on a floor which is a reinforced concrete structure;

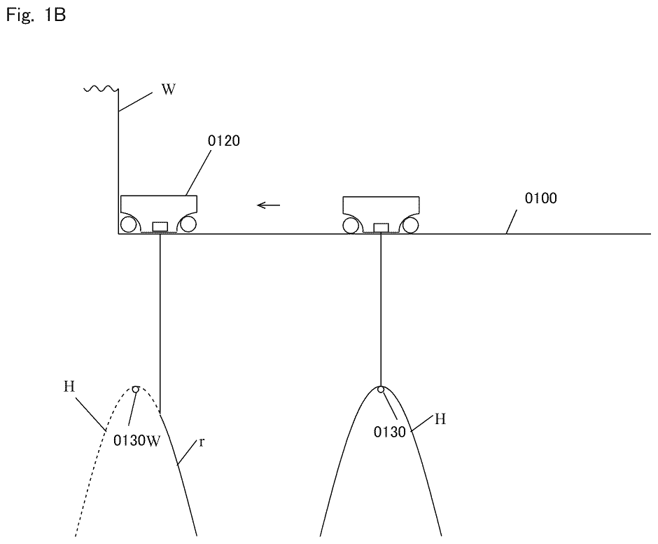

FIG. 2 is a diagram for explaining a hardware configuration;

FIG. 3A shows a schematic configuration of a device for estimating a radar invisible region of a reinforced concrete structure according to Embodiment 1;

FIG. 3B is a plan view of the floor which is the reinforced concrete structure;

FIG. 3C is a functional block diagram of the device for estimating the radar invisible region of the reinforced concrete structure according to Embodiment 1;

FIG. 3D is a schematic diagram for explaining a reason why a hyperbola waveform is formed on a two-dimensional image by radiation of an electromagnetic wave to the reinforcing bar of the reinforced concrete structure;

FIG. 3E is a diagram illustrating characteristics of the hyperbola waveform formed on the two-dimensional image by radiation of the electromagnetic wave to the reinforcing bar of the reinforced concrete structure;

FIG. 3F is a diagram similar to FIG. 3E illustrating characteristics of the hyperbola waveform formed on the two-dimensional image by the radiation of the electromagnetic wave to the reinforcing bar of the reinforced concrete structure;

FIG. 3G is a two-dimensional image schematically illustrating the hyperbola waveform related to the reinforcing bar buried in the vicinity of the wall acquired by performing the radar scanning on the floor which is the reinforced concrete structure;

FIG. 3H is a diagram for explaining points for estimating a hyperbola figure whose vertex is visible from data (curvature) of a skirt (waveform part) of the hyperbola waveform whose vertex is invisible by using an equation of a curve derived on the basis of the hyperbola figure;

FIG. 4 is a diagram illustrating a hardware configuration example of the device for estimating the radar invisible region of the reinforced concrete structure according to Embodiment 1;

FIG. 5 is a processing flowchart of the device for estimating the radar invisible region of the reinforced concrete structure according to Embodiment 1;

FIG. 6A is a functional block diagram of a device for estimating a radar invisible region of a reinforced concrete structure according to Embodiment 2;

FIG. 6B is a diagram illustrating a plurality of hyperbola waveforms which are radar exploration results acquired by a radar exploration result acquisition unit;

FIG. 6C is a diagram for explaining points of curve fitting of a hyperbola waveform whose vertex is visible to a hyperbola waveform whose vertex is invisible by the device for estimating the radar invisible region of the reinforced concrete structure according to Embodiment 2;

FIG. 6D is a chart illustrating a plurality of hyperbola figures made into a database in the device for estimating the radar invisible region of the reinforced concrete structure according to Embodiment 2;

FIG. 7 is a diagram illustrating a hardware configuration example of the device for estimating the radar invisible region of the reinforced concrete structure according to Embodiment 2;

FIG. 8 is a processing flowchart of the device for estimating the radar invisible region of the reinforced concrete structure according to Embodiment 2;

FIG. 9A is a functional block diagram of a device for estimating a radar invisible region of a reinforced concrete structure according to Embodiment 3;

FIG. 9B is a diagram illustrating a plurality of hyperbola waveforms, which are radar exploration results acquired by a radar exploration result acquisition unit;

FIG. 9C is a chart illustrating a plurality of hyperbola figures made into a database in the device for estimating the radar invisible region of the reinforced concrete structure according to Embodiment 3;

FIG. 9D is a diagram for explaining points of curve fitting of a hyperbola figure as a template to a hyperbola waveform whose vertex is invisible by the device for estimating the radar invisible region of the reinforced concrete structure according to Embodiment 3;

FIG. 10 is a diagram illustrating a hardware configuration example of the device for estimating the radar invisible region of the reinforced concrete structure according to Embodiment 3; and

FIG. 11 is a processing flowchart of the device for estimating the radar invisible region of the reinforced concrete structure according to Embodiment 3.

### DETAILED DESCRIPTION

Embodiments of a device for estimating a radar invisible region of a reinforced concrete structure according to the present invention will be now explained. The relationship between embodiments and claims is as follows. Embodiment 1 relates mainly to claims 1, 3, 6, 7, 9, 12, 13, 15, and 18, Embodiment 2 relates mainly to claims 2, 4, 8, 10, 14, and 16, and Embodiment 3 relates mainly to claims 5, 11, and 17.

It is to be noted that the present invention is not limited to these embodiments but can be embodied in various forms within a range not departing from the gist thereof.

<Hardware that May Configure the Present Invention>

The present invention is an invention using an electronic computer in principle but is realized by software, by hardware, or by cooperation of the software and the hardware. The hardware that realizes all or some of the configuration requirements of the present invention consists of a CPU, a memory, a bus, an input/output device, various peripheral devices, a user interface, and the like, which are basic components of a computer. The various peripheral devices include: a storage device; an interface of the Internet or the like; a device for the Internet or the like; a display; a keyboard; a mouse; a speaker; a camera; a video camera; a television; various sensors for grasping the production status in laboratories, factories, or the like (a flow rate sensor, a temperature sensor, a weight sensor, a liquid amount sensor, an infrared sensor, a shipping number counter, a packing number counter, a foreign matter inspection device, a defective product counter, a radiation inspection device, a surface condition inspection device, a circuit inspection device, a motion detector, a device for grasping work status of workers (by video, ID, PC workload, or the like), and the like); a CD device; a DVD device; a Blu-ray device; a USB memory; a USB memory interface; a removable-type hard disk; a common hard disk; a projector device; an SSD; a telephone; a fax; a copying machine; a printer; a movie editing device; various sensor devices; and the like.

Moreover, the system of the present invention does not necessarily have to be configured by one housing but may be configured by connecting a plurality of housings through communication. Moreover, the communication may be via LAN, WAN, Wi-Fi (registered trademark), Bluetooth (registered trademark), infrared communication, or ultrasonic communication, and a part of thereof may be installed across national borders. In addition, each of the plurality of housings may be operated by different entities or may be operated by one entity. It does not matter whether the operation entity of the system of the present invention is singular or plural. Moreover, the present invention can be also configured as a system including a terminal used by a third party and a terminal used by another third party besides the system of the present invention. Moreover, these terminals may also be installed across national borders. In addition, in addition to the system of the present invention and the terminal, a device used for registering related information of a third party or a related person, a device used for a database for recording contents of the registration, and the like may be prepared. These devices may be provided in the system of the present invention, or the system of the present invention may be configured so that these devices are provided outside the system of the present invention and the system of the present invention can utilize such information.

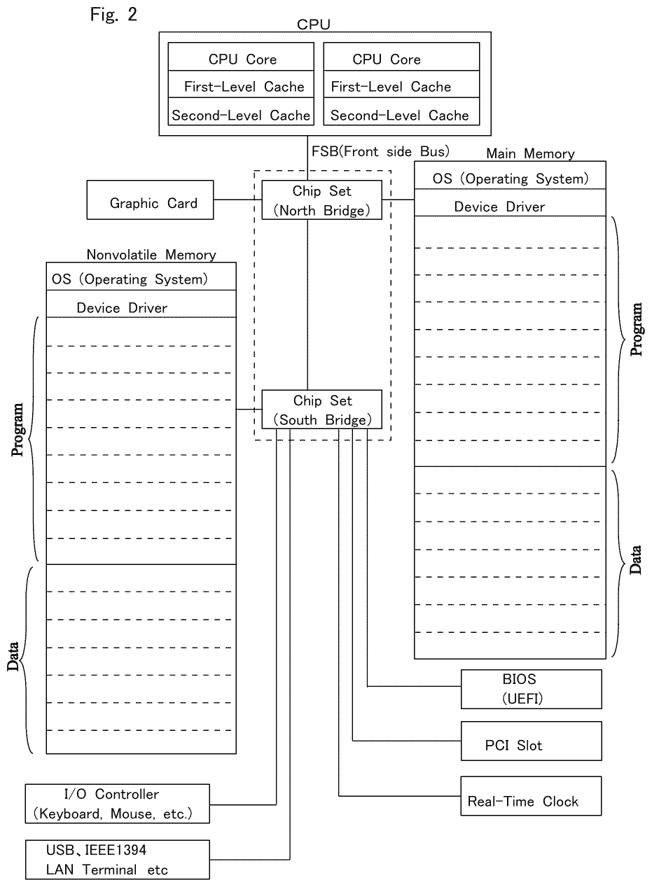

As shown in FIG. 2, the computer consists of a chip set, a CPU, a nonvolatile memory, a main memory, various buses, a BIOS, various interfaces, a real-time clock, and the like configured on a motherboard. They operate in cooperation with an operating system, device drivers, various programs, and the like. The various programs and various types of data configuring the present invention are configured to efficiently utilize these hardware resources for executing various types of processing.

### <Chip Set>

The “chip set” is a set of large-scale integrated circuits (LSI) which are mounted on a motherboard of a computer and in which functions of connecting an external bus of a CPU and standard buses for connecting a memory and peripheral devices, that is, bridge functions are integrated. There are a case where a two chip-set configuration is adopted and a case where a one chip-set configuration is adopted. A north bridge is provided on a side closer to the CPU and the main memory, while a south bridge is provided on a far side, i.e., a side of the interface with an external I/O at a relatively low speed.

### (North Bridge)

The north bridge includes a CPU interface, a memory controller, and a graphic interface. Most of the functions of the conventional north bridge may be borne by the CPU. The north bridge is connected to a memory slot of the main memory via a memory bus and is connected to a graphic card slot of a graphic card via a high-speed graphic bus (AGP, PCI Express).

### (South Bridge)

The south bridge is connected to a PCI interface (PCI slot) via a PCI bus and bears an I/O function with an ATA (SATA) interface, a USB interface, an Ethernet interface, and the like and a sound function. Incorporation of a circuit which supports a PS/2 port, a floppy disk drive, a serial port, a parallel port, and an ISA bus in which a high-speed operation is not needed or impossible will hinder the speeding up of the chip set itself. Therefore, it may be separated from a chip of the south bridge and placed in another LSI called a super I/O chip. A bus is used to connect the CPU (MPU) to the peripheral devices and the various control units. The bus is connected by the chip set. For speeding up, a channel structure may be employed in place of the memory bus used for connection with the main memory. A serial bus or a parallel bus can be employed as the bus. While the serial bus transfers data of one bit at a time, the parallel bus transmits original data itself or a plurality of bits cut out from the original data in a lump through a plurality of communication paths at the same time. A dedicated line for a clock signal is provided in parallel with a data line and executes synchronization of data demodulation on a reception side. GPIB, IDE/(parallel) ATA, SCSI, PCI, and the like are also used as buses for connecting the CPU (chip set) and external devices. Since there is a limit to speeding up, a serial bus may be used for a data line in an improved version of PCI, called PCI Express, or an improved version of parallel ATA, called serial ATA.

### <CPU>

The CPU sequentially reads, interprets, and executes instruction strings called programs on the main memory and outputs information consisting of signals also onto the main memory. The CPU functions as a center of executing calculations in the computer. It is to be noted that the CPU is consisted of a CPU core part as a center of the calculation and a peripheral part thereof, and a register, a cache memory, an internal bus for connecting the cache memory and the CPU core, a DMA controller, a timer, an interface with a connection bus to the north bridge, and the like are included inside the CPU. It is to be noted that a plurality of the CPU cores may also be provided in one CPU (chip). Moreover, in addition to the CPU, processing may be executed by a graphic interface (GPU) or an FPU.

### <Nonvolatile Memory>

### (HDD)

A hard disk drive is essentially configured by a magnetic disk, a magnetic head, and an arm on which the magnetic head is mounted. An SATA (ATA in the past) can be employed as an external interface. A highly functional controller such as an SCSI is used to support communication between hard disk drives. For example, when a file is to be copied to another hard disk drive, the controller can read a sector and transfer it to another hard disk drive for writing. At this time, the memory of a host CPU is not accessed. Therefore, there is no need to increase the load on the CPU.

It is to be noted that, as the nonvolatile memory, an SSD configured by an “NAND flash” may be employed together with the HDD or may be employed in place of the HDD.

### <Main Memory>

The CPU directly accesses and executes various programs on the main memory. The main memory is a volatile memory, and a DRAM is used. The program on the main memory is expanded from the nonvolatile memory onto the main memory upon receipt of a program activation instruction. Thereafter, the CPU continues to execute the program in accordance with various execution instructions and execution procedures in the program.

### <Operating System (OS)>

The operating system is used to manage resources on the computer to be used by applications, to manage various device drivers, and to manage the computer itself, which is hardware. In a small-sized computer, firmware may be used as an operating system.

### <Device Driver>

The device driver is a program which controls the hardware of the device to enable the various devices attached to a computer to be used by a user or an application via the operating system.

### <BIOS>

The BIOS causes the CPU to execute the procedure to start up the hardware of the computer and operate the operating system and is most typically the first piece of hardware that the CPU reads when the CPU receives the activation instruction for the computer. An address of the operating system stored in the disk (nonvolatile memory) is described on the BIOS, and the operating system is sequentially expanded in the main memory by the BIOS expanded in the CPU and becomes operational. It is to be noted that the BIOS also has a check function for checking presence or absence of the various devices connected to the bus. The results of the check are stored in the main memory and made available to the operating system as appropriate. It is to be noted that the BIOS may be configured to check external devices and the like.

### <I/O Controller>

The I/O controller is used for connection with external devices. A USB connector is an example thereof.

### <USB, IEEE1394 Connector, LAN Terminal, and the Like>

A USB, an IEEE1394 connector, a LAN terminal, and the like are the most typical communication standard interfaces.

The above is a configuration that is commonly used in the description of the hardware configuration in all embodiments in this specification.

### Embodiment 1

This Embodiment relates mainly to claims 1, 3, 6, 7, 9, 12, 13, 15, and 18.

### Embodiment 1: Device for Estimating Radar Invisible Region of Reinforced Concrete Structure, Outline

A device for estimating a radar invisible region of a reinforced concrete structure according to this embodiment causes an electromagnetic wave radar to scan the reinforced concrete structure, collects position data of a reinforcing bar disposed inside the reinforced concrete structure, and, as described in detail in the following paragraphs 0057 to 0059, acquires so-called hyperbola information, which is waveform information of a reflected wave relating to the reinforcing bar on the basis of the position data of this reinforcing bar. Then, in this estimation device, in a case where the acquired hyperbola information includes waveform information of a waveform part in which a vertex is invisible, the position of the reinforcing bar is estimated on the basis of the waveform information of the waveform part in which a vertex is invisible.

This device for estimating the radar invisible region of the reinforced concrete structure has, for example, an effect that, when a hyperbola waveform is to be acquired by causing the electromagnetic wave radar to scan the floor or the like, which is the reinforced concrete structure, the position of the reinforcing bar buried in this floor in the vicinity of the wall can be estimated even if the hyperbola waveform related to the reinforcing bar in the floor in the vicinity of the wall is a waveform in which only a skirt part thereof is visible but a vertex is invisible.

### Embodiment 1: Device for Estimating Radar Invisible Region of Reinforced Concrete Structure, Configuration, Corresponding Mainly to Claims 1, 3, 6, 7, 9, 12, 13, 15, and 18

### <Reinforced Concrete Structure>

The reinforced concrete structure as a target of the estimation by the device for estimating the radar invisible region of the reinforced concrete structure according to the present invention includes pillars, beams, floors, walls, bases, and the like of a building, as well as bridges, tunnels, and the like.

### <Estimation Device, and the Like>

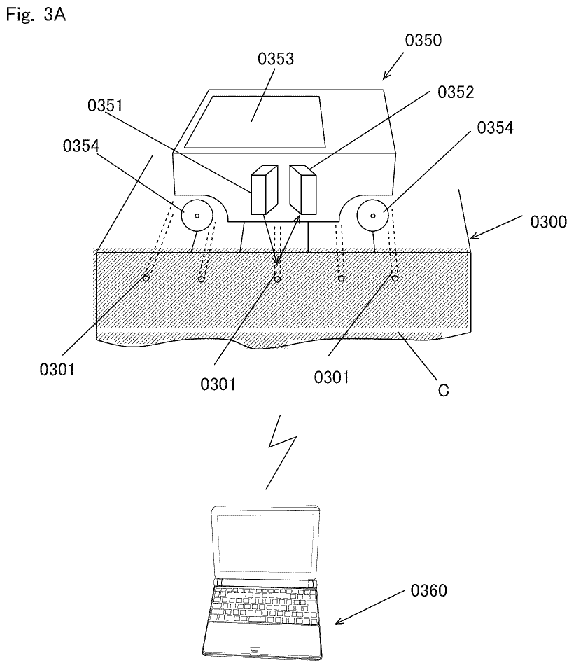

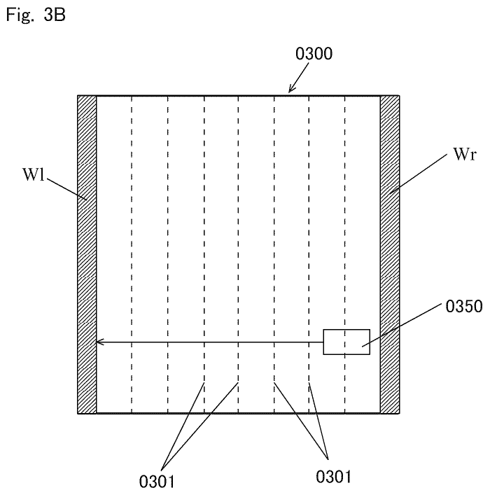

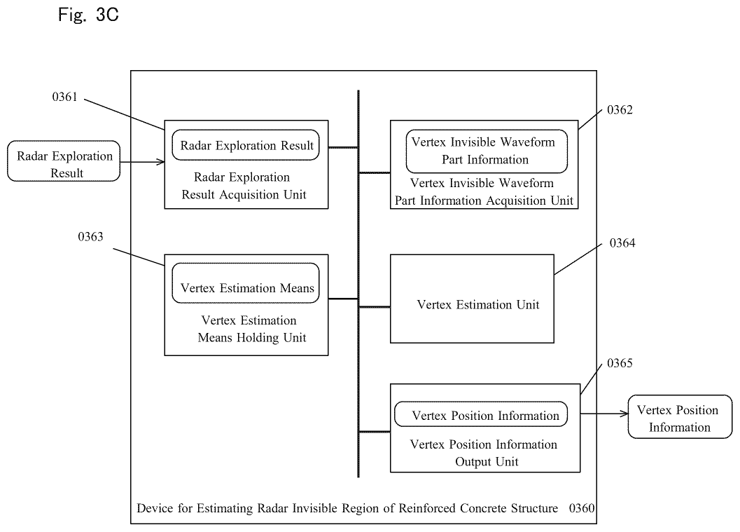

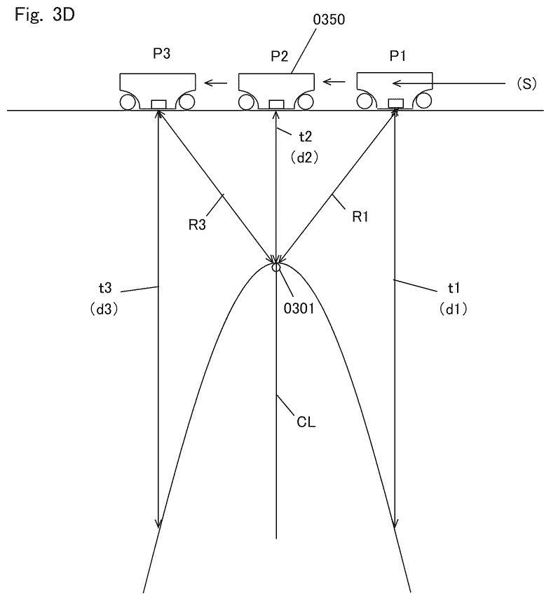

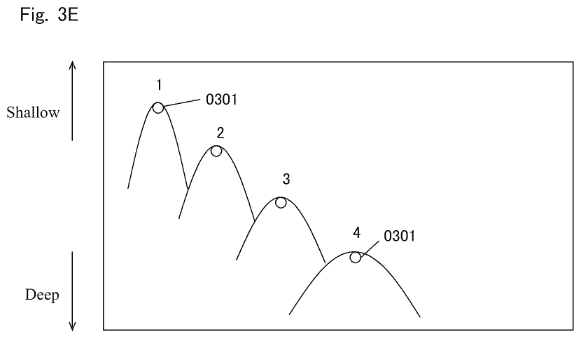

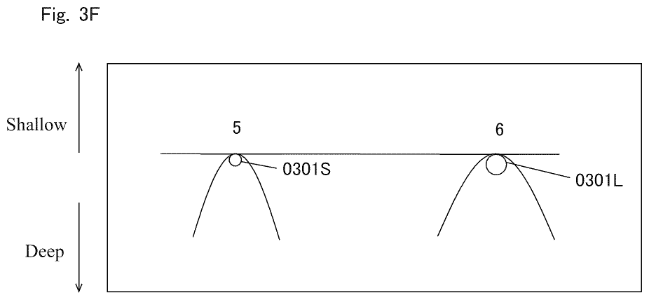

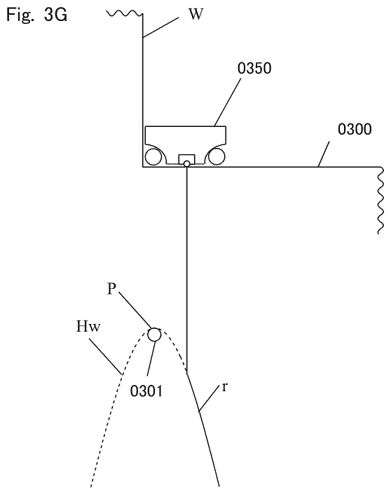

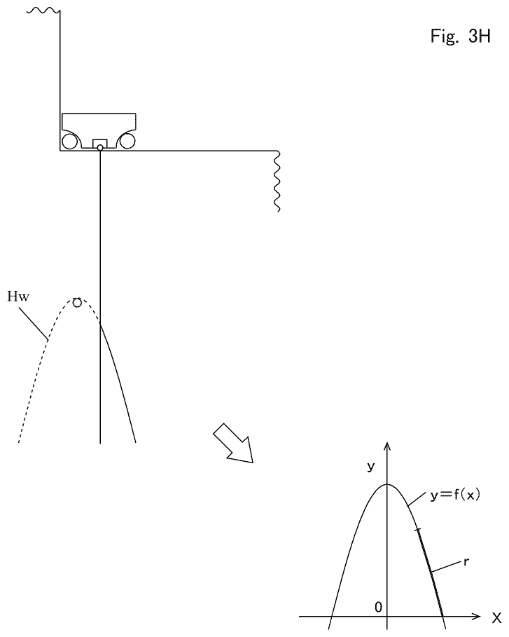

As shown in FIG. 3A, the device 0360 for estimating the radar invisible region of the reinforced concrete structure of this embodiment is a computer such as a laptop computer and collects position data of a plurality of reinforcing bars 0301 buried and arranged in a floor 0300 of a reinforced concrete structure such as a building by using an electromagnetic wave radar 0350 having a carriage-like shape, which will be described later. Although it is common in the reinforced concrete structure that a plurality of reinforcing bars are disposed in a direction substantially orthogonal to the plurality of reinforcing bars 0301 so as to form upper and lower two layers of reinforcing bars, the reinforcing bars substantially orthogonal to the reinforcing bars 0301 are omitted for simplification in FIG. 3A. Moreover, the present invention can be naturally applied not only to a case where the reinforcing bars are disposed in one layer or in upper and lower two layers inside the reinforced concrete structure but also to a case where the reinforcing bars are disposed in three or more layers.

In this Embodiment, the device 0360 for estimating a radar invisible region and the electromagnetic wave radar 0350 are configured to transmit and receive data via a wireless LAN. The data transmission and reception via the wireless LAN is performed in a state where the electromagnetic wave radar is not radiated. The data transmission and reception may also be configured to be performed via a wire or via a memory such as a USB memory.

### <Radar Exploration>

The radar exploration in the device for estimating the radar invisible region of the reinforced concrete structure according to the present invention is exploration using an electromagnetic wave, and a depth or a position of a reinforcing bar or a foreign substance inside the reinforced concrete structure is identified by radiating high-frequency electromagnetic waves into the reinforced concrete structure and receiving reflected waves.

### <Electromagnetic Wave Radar>

As shown in FIG. 3A, the electromagnetic wave radar 0350 of a carriage-like shape includes: a transmission antenna 0351 which starts at a predetermined scanning start position set on the floor 0300 and radiates electromagnetic waves to the inside of the floor 0300 while moving; a receiving antenna 0352 which receives a reflected wave of the radiated electromagnetic wave from the reinforcing bar 0301 inside the floor 0300 while moving; and a radar screen 0353 which displays a position according to the time from when the electromagnetic waves are radiated from the transmission antenna 0351 to when they are reflected by the reinforcing bar and received by the receiving antenna 0352 (two arrows in opposing directions in FIG. 3A indicate electromagnetic waves radiated to the reinforcing bar 0301 at a center of the FIG. 3A and the electromagnetic waves reflected back, respectively).

### <Distance Measurement and the Like Related to Electromagnetic Wave Radar>

In this case, a distance meter, not shown, such as a linear encoder or a rotary encoder is incorporated in a wheel 0354 of the electromagnetic wave radar 0350 so that a distance from a scanning start position to a current position in the radar scanning of the floor 0300 is known. It is to be noted that, since a moving speed when the radar scanning is performed by the electromagnetic wave radar 0350 is not involved in the grasping of a disposition state of the reinforcing bars 0301, the electromagnetic wave radar 0350 may be manually moved in the radar scanning or may be self-driven at a constant speed by a motor, for example.

### <Moving Methods and the Like of Electromagnetic Wave Radar>

Here, in a case where the electromagnetic wave radar 0350 is self-driven, movement may be made by using a quasi-zenith satellite system (Michibiki) for the radar scanning outdoors such as on a rooftop of a building or a bridge. On the other hand, for the radar scanning indoors such as on a floor surface of a building as described above, for example, a plurality of beacons may be disposed inside the building and a receiving terminal for the beacons may be mounted on the electromagnetic wave radar 0350 so that the electromagnetic wave radar 0350 is guided by the plurality of beacons and moved.

### <Radar Exploration Points of Electromagnetic Wave Radar>

When the floor 0300 is radar-scanned using the electromagnetic wave radar 0350 as above, the electromagnetic wave radar 0350 is moved on the floor 0300 in a direction orthogonal to the reinforcing bar 0301. When the reinforced concrete structure is the floor 0300 of a building as in this embodiment, as shown in a plan view of the floor in FIG. 3B, a plurality of the reinforcing bars 0301 are generally arranged in parallel to walls Wl and Wr located on left and right of the floor 0300. In consideration of this, the electromagnetic wave radar 0350 is moved from a position in the vicinity of the wall Wr on the right side in the figure toward the wall Wl on the left side in the figure, for example, so that the position of each of the plurality of reinforcing bars 0301 arranged side by side in a left-right direction of the floor 0300 in the figure is grasped.

### Embodiment 1: Device Estimating for Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Entire

As shown in FIG. 3C, the device 0360 for estimating a radar invisible region of a reinforced concrete structure of this embodiment has a radar exploration result acquisition unit 0361, a vertex invisible waveform part information acquisition unit 0362, a vertex estimation means holding unit 0363, a vertex estimation unit 0364, and a vertex position information output unit 0365.

### Embodiment 1: Device Estimating for Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Radar Exploration Result Acquisition Unit

The “radar exploration result acquisition unit 0361” performs radar scanning by the electromagnetic wave radar in the direction orthogonal to the reinforcing bars in order to acquire, as a radar exploration result, a hyperbola waveform (information) which is waveform information of a reflected wave relating to each of the plurality of reinforcing bars.

### Embodiment 1: Waveform Information Acquired by Radar Exploration Result Acquisition Unit (Hyperbola Waveform)

Here, as shown in the schematic diagram combining a two-dimensional image of a radar screen with a cross section of a buried reinforcing bar in FIG. 3D, when performing the radar scanning by moving the electromagnetic wave radar 0350 from a scanning start point S in a direction (left-right directions in the figure) orthogonal to the direction in which the reinforcing bars 0301 are arranged, the electromagnetic wave radiated from the transmission antenna advances with spreading out in forward and backward directions. Therefore, at a point of a distance P1 from the scanning start point S measured by the distance meter before the electromagnetic wave radar 0350 passes immediately above the reinforcing bar 0301, for example, the electromagnetic wave radar 0350 receives a diagonal reflected wave R1 from the front reinforcing bar 0301. At this time, a time t1 from when the electromagnetic wave is radiated at the point P1 to when it returns as the reflected wave R1, or a distance d1 from the electromagnetic wave radar 0350 to the reinforcing bar 0301 which can be obtained in proportion to the time t1 is displayed immediately below the point P1 on the radar screen of the electromagnetic wave radar 0350. Then at a point of a distance P2 from the scanning start point S measured by the distance meter when the electromagnetic wave radar 0350 is immediately above the reinforcing bar 0301, a time t2 from the electromagnetic wave is radiated at this point P2 to when it returns as the reflected wave, or a distance d2 from the electromagnetic wave radar 0350 to the reinforcing bar 0301 which can be obtained in proportion to the time t2 becomes the shortest, and this time or distance is displayed as a position of the reinforcing bar corresponding to the point P2 on the radar screen. Thereafter, when the electromagnetic wave radar 0350 passes a point of a distance P3 from the scanning start point S measured by the distance meter, the electromagnetic wave radar 0350 receives a diagonal reflected wave from the passed reinforcing bar 0301, and a time t3 from when the electromagnetic wave is radiated at the point P3 to when it returns as a reflected wave R3, or a distance d3 from the electromagnetic wave radar 0350 to the reinforcing bar 0301 which can be obtained in proportion to this time t3 is displayed immediately below the point P3 on the radar screen of the electromagnetic wave radar 0350.

### <Vertex (Peak) of Hyperbola Waveform and Reinforcing Bar>

As the result of the radar exploration as described above, the positions of the reinforcing bar at the points of the distances P1 to P3 from the scanning start point S measured by the distance meter are traced and displayed on the radar screen as a symmetrical mountain-shaped waveform with a vertical line passing the reinforcing bar 0301 as a center, that is, a hyperbola waveform (information) of the reinforcing bar 0301. On the basis of a vertex (peak) of this hyperbola waveform of the reinforcing bar 0301, it can be recognized that the reinforcing bar 0301 is located on a center line CL of the symmetric hyperbola waveform. That is, information on an arrangement position of the reinforcing bar 0301 in a horizontal direction of the floor 0300 can be acquired.

It is to be noted that, the device can be configured to emit a passing sound or light which notifies the surroundings of the passage of this electromagnetic wave radar 0350 when the electromagnetic wave radar 0350 passes through the point P2 immediately above the reinforcing bar 0301 where the distance d2 from the reinforcing bar 0301 to the electromagnetic wave radar 0350 is the shortest.

### <Characteristics of Hyperbola Waveform>

As shown in FIG. 3E, this hyperbola waveform becomes a hyperbola waveform 1 having a mountain shape with a sharp vertex part when a depth of the reinforcing bar 0301 is shallow, and the deeper the depth of the reinforcing bar 0301 with the same diameter becomes, this hyperbola waveform becomes hyperbola waveforms 2, 3, 4 having a mountain shape with a gentler vertex part. Moreover, in a case where diameters of the reinforcing bars buried at the same depth positions are different from each other, as shown in FIG. 3F, a hyperbola waveform related to a thin reinforcing bar 0301S becomes a hyperbola waveform 5 with a sharp vertex part, while a hyperbola waveform related to a thick reinforcing bar 0301L becomes a hyperbola waveform 6 with a gentle vertex part.

### Embodiment 1: Device Estimating for Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Vertex Invisible Waveform Part Information Acquisition Unit

The “vertex invisible waveform part information acquisition unit 0362” functions so as to acquire waveform information of a waveform part whose vertex is invisible in the hyperbola waveform which is a radar exploration result acquired by the radar exploration result acquisition unit 0361.

### <Waveform Information of Waveform Part Whose Vertex is Invisible>

The “waveform information of a waveform part whose vertex is invisible” refers to waveform information of a hyperbola waveform in a state where a vertex (peak) is missing and only a skirt part can be recognized of waveform information acquired by the aforementioned radar exploration.

### <Specific Example of Waveform Information of Waveform Part Whose Vertex is Invisible>

Specifically, as shown in FIG. 3G, for example, when the floor 0300, which is a reinforced concrete structure, is blocked by the wall W, advance of the electromagnetic wave radar 0350 is restricted at the wall. Thus, when the reinforcing bar 0301 is buried in the floor 0300 in the vicinity of the wall W, even if the radar scanning is performed up to the vicinity of the wall W, information acquired in the vicinity of this wall W becomes a partial waveform, i.e., only the skirt r (solid line part) of a hyperbola waveform Hw and its vertex p (broken line part) is invisible.

The “vertex invisible waveform part information acquisition unit 0362” acquires the waveform information of the skirt r (waveform part) of the hyperbola waveform Hw whose vertex is invisible as described above.

### Embodiment 1: Device Estimating for Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Vertex Estimation Means Holding Unit

The “vertex estimation means holding unit 0363” has a function of holding the estimation means for estimating the vertex position of the vertex invisible waveform part on the basis of the vertex invisible waveform part information acquired by the vertex invisible waveform part information acquisition unit 0362.

### Embodiment 1: Configuration, Functional Block, Vertex Estimation Means

This vertex estimation means holding unit 0363 holds vertex estimation means for calculating a hyperbola figure acquired by substituting data (curvature) of a skirt (waveform part) of a hyperbola waveform whose vertex is invisible for an equation y=f(x) of a curve derived on the basis of the hyperbola figure. In this case, “x” in the curve equation y=f(x) is a distance from the scanning start point measured by the distance meter.

### Embodiment 1: Device for Estimating Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Vertex Estimation Unit

The “vertex estimation unit 0364” functions so as to estimate a vertex position of the vertex invisible waveform part on the basis of the vertex invisible waveform part information and the vertex estimation means.

### Embodiment 1: Configuration, Functional Block, Estimation of Vertex Position by Vertex Estimation Unit

In this vertex estimation unit 0364, as shown in FIG. 3H, the hyperbola figure is acquired by substituting the curvature of the skirt (waveform part) r of the hyperbola waveform whose vertex is invisible for the equation y=f(x) derived on the basis of the hyperbola figure, which is a quadratic curve. Then, on the display in the device for estimating the radar invisible region, which is a computer such as a laptop personal computer, or on the radar screen of the electromagnetic wave radar, the acquired hyperbola figure is superimposed on a hyperbola waveform, which is the vertex invisible waveform part information, in which only the skirt is visible but the vertex is invisible, so as to estimate the vertex position of the vertex invisible waveform part.

### Embodiment 1: Device for Estimating Radar Invisible Region of Reinforced Concrete Structure, Configuration, Corresponding Mainly to Claims 1, 3, 6, 7, 9, 12, 13, 15, and 18

### Embodiment 1: Configuration, Functional Block, Vertex Position Information Output Unit

The “vertex position information output unit 0365” outputs the vertex position information estimated by the vertex estimation unit 0364. That is, the information on the position in the horizontal direction of the reinforcing bar 0301 buried in the floor 0300 in the vicinity of the wall is output.

### Embodiment 1: Device for Estimating Radar Invisible Region of Reinforced Concrete Structure, Hardware Configuration, Corresponding Mainly to Claims 1, 3, 6, 7, 9, 12, 13, 15, and 18

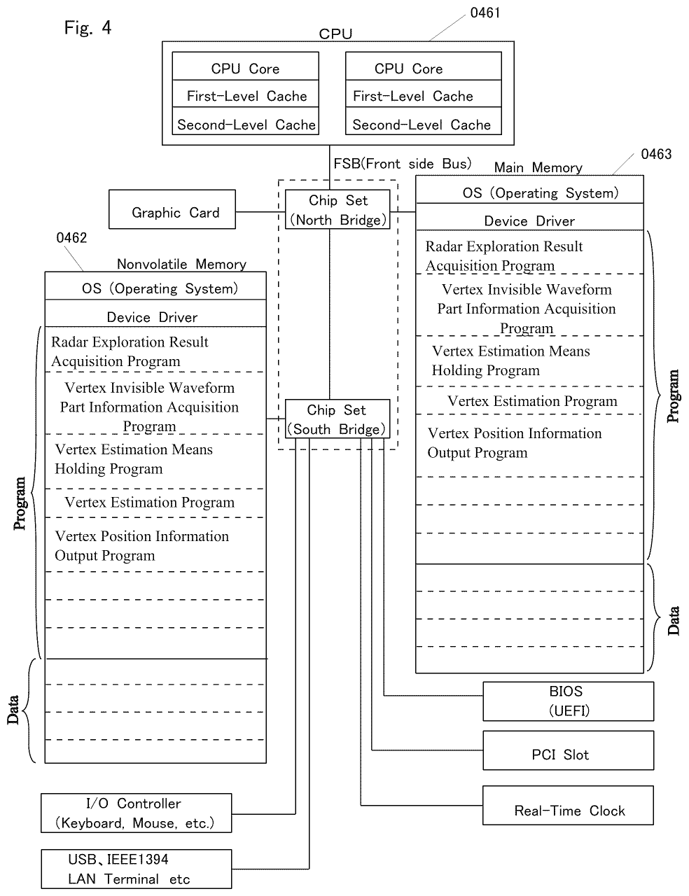

As shown in FIG. 4, a device 0460 for estimating a radar invisible region of a reinforced concrete structure according to this embodiment consists of a CPU 0461, a nonvolatile memory 0462 such as an HDD and a ROM, a main memory 0463 such as a D-RAM, and an interface. The nonvolatile memory 0462 stores a radar exploration result acquisition program, a vertex invisible waveform part information acquisition program, a vertex estimation means holding program, a vertex estimation program, and a vertex position information output program. The data is information on a current signal and a phase angle, and these programs and data are read into a holding area of the main memory 0463 and executed in an operation area. Moreover, the interface includes a specific low-power radio and the like.

### Embodiment 1: Device for Estimating Radar Invisible Region of Reinforced Concrete Structure, Flow of Processing, Corresponding Mainly to Claims 1, 3, 6, 7, 9, 12, 13, 15, and 18

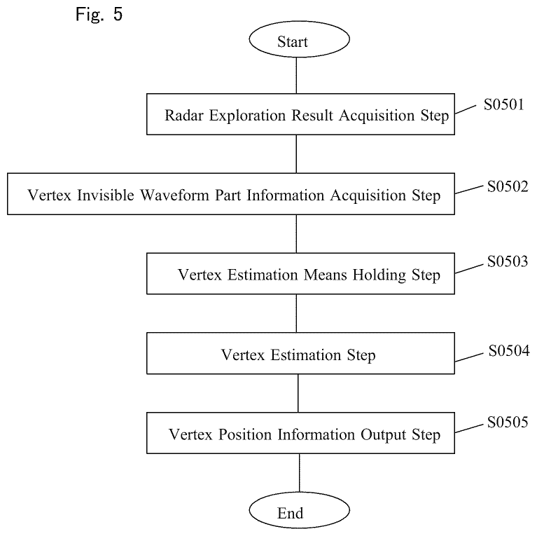

In the device for estimating the radar invisible region of the reinforced concrete structure according to this embodiment, as shown in FIG. 5, a radar exploration result acquisition step S0501 is executed for acquiring a radar exploration result of the reinforced concrete structure.

Subsequently, a vertex invisible waveform part information acquisition step S0502 is executed for acquiring the waveform information of the waveform part of the hyperbola waveform whose vertex is invisible from the acquired radar exploration result.

Subsequent to the above, the vertex estimation means holding step S0503 is executed for holding the estimation means for estimating the vertex position of the vertex invisible waveform part on the basis of the vertex invisible waveform part information.

Then, the vertex estimation step S0504 is executed for estimating the vertex position of the vertex invisible waveform part on the basis of the data (curvature) of the skirt (waveform part) of the hyperbola waveform whose vertex is invisible, which is the vertex invisible waveform part information, and a quadratic equation y=f(x) derived on the basis of the hyperbola figure, which is the vertex estimation means. Thereafter, the vertex position information output step S0505 is executed for outputting the estimated vertex position information.

### Embodiment 1: Device for Estimating Radar Invisible Region of Reinforced Concrete Structure, Effects, Corresponding Mainly to Claims 1, 3, 6, 7, 9, 12, 13, 15, and 18

The device for estimating the radar invisible region of the reinforced concrete structure according to this embodiment has, for example, an effect that, when a hyperbola waveform is acquired by causing the radar to scan the floor, the wall, the pillar, the ceiling, or the like, which is the reinforced concrete structure, the positions of the reinforcing bars in the vicinity of the wall can be estimated even if the hyperbola waveform related to the reinforcing bars buried in the floor or the like in the vicinity of the wall is a waveform in which only a skirt part thereof is visible but a vertex is invisible.

### Embodiment 2

This Embodiment relates mainly to claims 2, 4, 8, 10, 14, and 16.

### Embodiment 2: Device for Estimating Radar Invisible Region of Reinforced Concrete Structure, Outline

This Embodiment is based on Embodiment 1, and when there is waveform information of a waveform part whose vertex is invisible together with the waveform information in which the vertex is visible in the hyperbola information acquired by causing the electromagnetic wave radar to scan the reinforced concrete structure, a device for estimating a radar invisible region of a reinforced concrete structure according to this embodiment estimates a vertex position of the invisible waveform part by using the waveform information in which the vertex is visible so as to estimate a position of the reinforcing bar for which the hyperbola information in which the vertex is invisible was acquired.

The device for estimating the radar invisible region of the reinforced concrete structure according to this embodiment has, for example, an effect that, when a hyperbola waveform is to be acquired by causing the electromagnetic wave radar to scan the floor or the like, which is the reinforced concrete structure, similarly to the estimation device according to the above-described embodiment, the position of the reinforcing bar in this floor in the vicinity of the wall can be estimated even if the hyperbola waveform of the reinforcing bar buried in the floor in the vicinity of the wall is a waveform in which only the skirt part is visible, but the vertex is not visible.

### Embodiment 2: Device for Estimating Radar Invisible Region of Reinforced Concrete Structure, Configuration, Corresponding Mainly to Claims 2, 4, 8, 10, 14, and 16

### Embodiment 2: Configuration, Functional Block, Entire

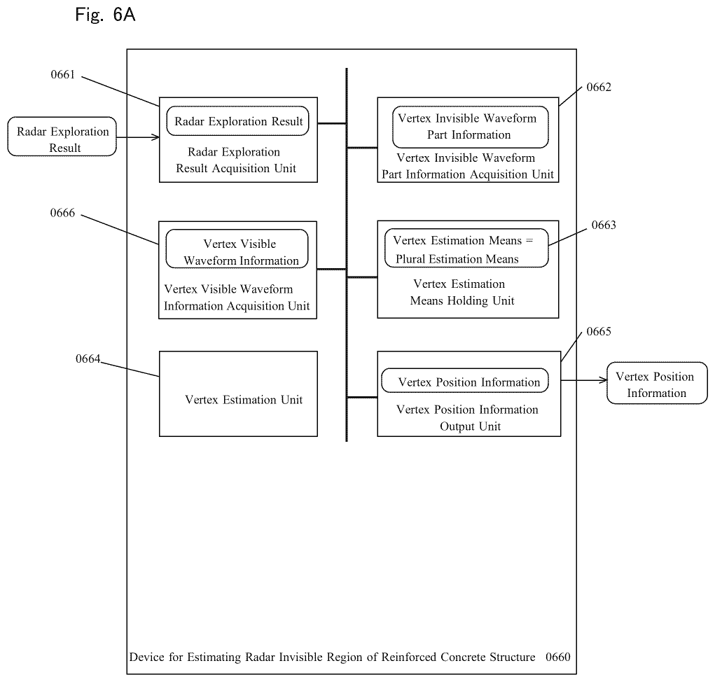

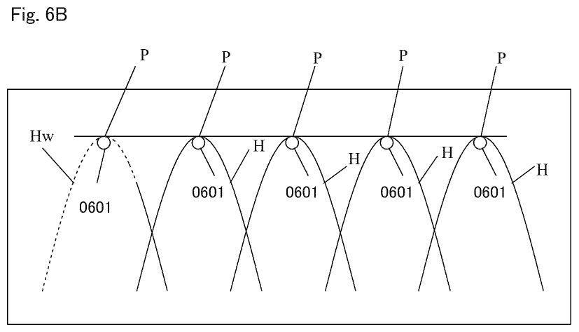

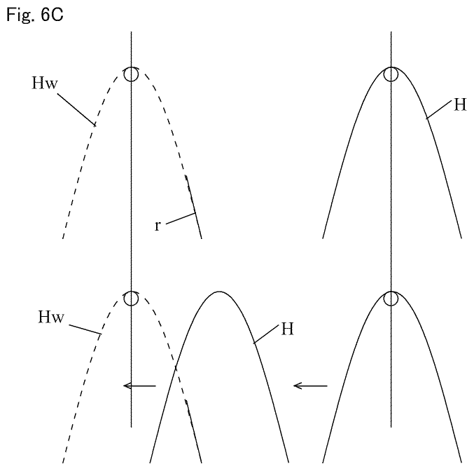

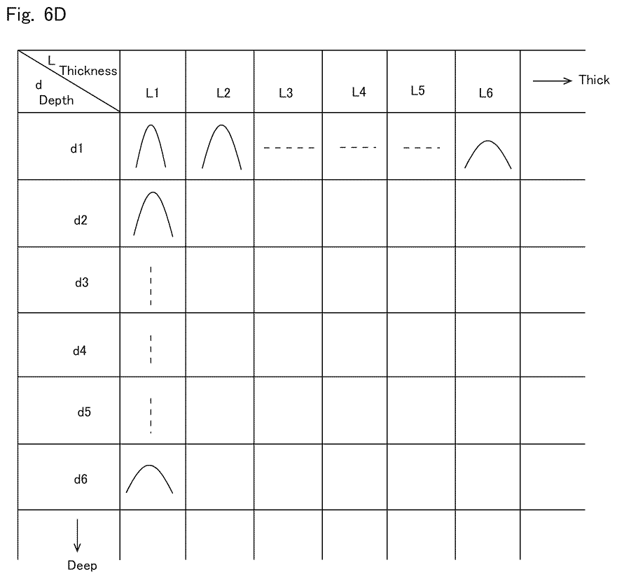

As shown in FIG. 6A, a device 0660 for estimating a radar invisible region of a reinforced concrete structure of this embodiment has a radar exploration result acquisition unit 0661, a vertex invisible waveform part information acquisition unit 0662, a vertex estimation means holding unit 0663, a vertex estimation unit 0664, and a vertex position information output unit 0665, as well as a vertex visible waveform information acquisition unit 0666.

### Embodiment 2: Device for Estimating Radar Invisible Region of Reinforced Concrete Structure, Configuration, Corresponding Mainly to Claims 2, 4, 8, 10, 14, and 16

### Embodiment 2: Configuration, Functional Block, Vertex Visible Waveform Information Acquisition Unit

The “vertex visible waveform information acquisition unit 0666” functions so as to acquire the waveform information of the vertex visible waveform.

### <Vertex Visible Waveform Information>

The vertex visible waveform information is waveform information of a hyperbola waveform acquired by the radar exploration in a state where a vertex (peak) can be recognized.

### Specific Example of Vertex Visible Waveform Information

Specifically, as shown on the radar screen in FIG. 6B, a symmetric mountain-shaped waveform whose vertex p related to the reinforcing bar 0601 is visible, i.e., information on the arrangement positions of a plurality of the reinforcing bars 0601 in the horizontal direction of the floor when radar-scanning the floor by using the electromagnetic wave radar, for example, is acquired from the hyperbola waveform, which is a radar exploration result acquired by the radar exploration result acquisition unit 0661.

### Embodiment 2: Configuration, Functional Block, Vertex Estimation Means Holding Unit

<When a Hyperbola Waveform, which is a Radar Exploration Result, is Vertex Visible Waveform Information>

In this Embodiment, the “vertex estimation means holding unit 0663” holds plural estimation means as vertex estimation means for extracting a hyperbola waveform (hyperbola waveform suitable for curve fitting described later) having a skirt which can be superimposed on a skirt (waveform part) of the hyperbola waveform whose vertex is invisible from among the plurality of hyperbola waveforms acquired by the vertex visible waveform information acquisition unit 0666.

As described above, in the case of the floor 0300 which is a reinforced concrete structure, a plurality of reinforcing bars with the same diameter including the reinforcing bars in the vicinity of the wall are generally arranged on the same level and in parallel to each other. In consideration of this, in such a case, as shown on the radar screen in FIG. 6B, it is assumed that a plurality of hyperbola waveforms H with the same shape are acquired by the vertex visible waveform information acquisition unit 0666. Therefore, the plural estimation means in the vertex estimation means holding unit 0663 extracts any one of the hyperbola waveforms from the plurality of hyperbola waveforms H with the same shape.

### Embodiment 2: Device for Estimating Radar Invisible Region of Reinforced Concrete Structure, Configuration, Corresponding Mainly to Claims 2, 4, 8, 10, 14, and 16

### Embodiment 2: Configuration, Functional Block, Vertex Estimation Unit

In this Embodiment, in the “vertex estimation unit 0664”, on the display in the device for estimating the radar invisible region, which is a computer such as a laptop personal computer, or on the radar screen of the electromagnetic wave radar, a hyperbola waveform H whose vertex is visible, which is one piece of the vertex visible waveform information acquired by the plural estimation means of the vertex estimation means holding unit 0663 and a hyperbola waveform Hw in which only the skirt r is visible and the vertex is invisible, which is the vertex invisible waveform part information, are displayed as shown on an upper part in FIG. 6C. Then, as shown on a lower part of FIG. 6C, the hyperbola waveform H whose vertex is visible is moved in an arrow direction and curve-fitted to the hyperbola waveform Hw whose vertex is invisible so as to estimate the vertex position of the vertex invisible waveform part.

### Embodiment 2: Device for Estimating Radar Invisible Region of Reinforced Concrete Structure, Configuration, Corresponding Mainly to Claims 2, 4, 8, 10, 14, and 16

### <Another Specific Example of Information on Waveform Whose Vertex is Visible>

Here, in addition to the hyperbola waveform (vertex visible waveform information), which is the radar exploration result as described above, the waveform information of the vertex visible waveform acquired by the vertex visible waveform information acquisition unit 0666 from in the hyperbola waveform, which is the radar exploration result acquired by the radar exploration result acquisition unit 0661, includes a plurality of hyperbola figures made into a database on the basis of the aforementioned “characteristics that steepness of the vertex of the mountain shape in the hyperbola waveform changes in accordance with an buried depth of the reinforcing bar and a thickness of the reinforcing bar” as shown in FIG. 6D.

### Embodiment 2: Device for Estimating Radar Invisible Region of Reinforced Concrete Structure, Configuration, Corresponding Mainly to Claims 2, 4, 8, 10, 14, and 16

<A Case where a Plurality of Hyperbola Figures, which is Made into a Database, is Vertex Visible Waveform Information>

As described above, when the plurality of hyperbola figures made into a database are considered as the vertex visible waveform information in the vertex estimation means holding unit 0663, the plural estimation means is held as the vertex estimation means for extracting the hyperbola figure having a skirt which can be superimposed on the skirt of the hyperbola waveform whose vertex is invisible (waveform part) from among the plurality of hyperbola figures made into the database shown in FIG. 6D.

Then, when the plurality of hyperbola figures made into the database are considered as the vertex visible waveform information in the “vertex estimation unit 0664”, the hyperbola figure having the skirt which can be superimposed on the skirt of the hyperbola waveform whose vertex is invisible (waveform part) is extracted from the plurality of hyperbola figures made into the database shown in FIG. 6D. Then, on the display in the device for estimating the radar invisible region, which is a computer such as a laptop personal computer, or on the radar screen of the electromagnetic wave radar, the extracted hyperbola figure is curve-fitted to the hyperbola waveform in which only the skirt, which is the vertex invisible waveform part information, is visible and the vertex is invisible, similarly to the aforementioned hyperbola waveform Hw so as to estimate the vertex position of the vertex invisible waveform part.

### Embodiment 2: Device for Estimating Radar Invisible Region of Reinforced Concrete Structure, Hardware Configuration, Corresponding Mainly to Claims 2, 4, 8, 10, 14, and 16

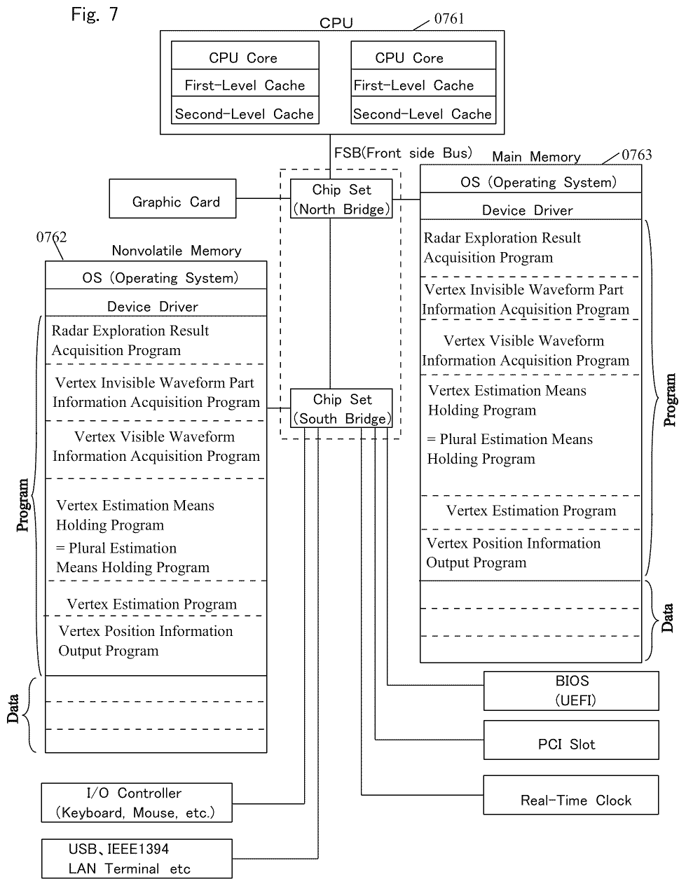

As shown in FIG. 7, a device 0760 for estimating a radar invisible region of a reinforced concrete structure according to this embodiment consists of a CPU 0761, a nonvolatile memory 0762 such as an HDD or a ROM, a main memory 0763 such as a D-RAM, and an interface. The nonvolatile memory 0762 stores a radar exploration result acquisition program, a vertex invisible waveform part information acquisition program, a vertex visible waveform information acquisition program, a vertex estimation means holding program, a vertex estimation program, and a vertex position information output program, and the plural estimation means as the vertex estimation means is used in the vertex estimation means holding program. The data is information on a current signal and a phase angle, and these programs and data are read into a holding area of the main memory 0763 and executed in an operation area. Moreover, the interface includes a specific low-power radio and the like.

### Embodiment 2: Device for Estimating Radar Invisible Region of Reinforced Concrete Structure, Flow of Processing, Corresponding Mainly to Claims 2, 4, 8, 10, 14, and 16

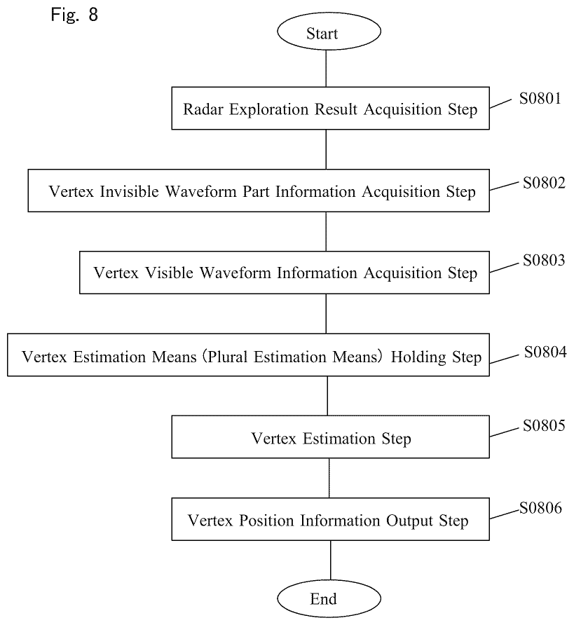

In the device for estimating the radar invisible region of the reinforced concrete structure according to this embodiment, as shown in FIG. 8, a radar exploration result acquisition step S0801 is executed for acquiring a radar exploration result of the reinforced concrete structure.

Subsequently, a vertex invisible waveform part information acquisition step S0802 is executed for acquiring the waveform information of the waveform part of the hyperbola waveform whose vertex is invisible from the acquired radar exploration result. Then, the vertex visible waveform information acquisition step S0803 is executed for acquiring the waveform information of the vertex visible waveform.

Subsequent to the above, a vertex estimation means holding step S0804 is executed for holding the plural estimation means as estimation means for estimating the vertex position of the vertex invisible waveform part on the basis of the vertex visible waveform information and the vertex invisible waveform part information.

Then, a vertex estimation step S0805 is executed for estimating the vertex position of the vertex invisible waveform part on the basis of the vertex visible waveform information, the vertex invisible waveform part information, and the plural estimation means as the vertex estimation means. Thereafter, the vertex position information output step S0806 is executed for outputting the estimated vertex position information.

### Embodiment 2: Device for Estimating Radar Invisible Region of Reinforced Concrete Structure, Effects, Corresponding Mainly to Claims 2, 4, 8, 10, 14, and 16

The device for estimating the radar invisible region of the reinforced concrete structure according to this embodiment has, for example, an effect that, when a hyperbola waveform is acquired by causing the radar to scan the floor, the wall, the pillar, the ceiling, or the like, which is the reinforced concrete structure, the positions of the reinforcing bars in the vicinity of the wall can be estimated even if the hyperbola waveform related to the reinforcing bars buried in the floor or the like in the vicinity of the wall is a waveform in which only a skirt part thereof is visible but a vertex is invisible.

### Embodiment 3

This Embodiment relates mainly to claims 5, 11, and 17.

### Embodiment 3: Device for Estimating Radar Invisible Region of Reinforced Concrete Structure, Outline

This Embodiment is based on Embodiments 1, 2, and when there is waveform information of a waveform part whose vertex is invisible together with the waveform information in which the vertex is visible in the hyperbola information acquired by causing the electromagnetic wave radar to scan in the reinforced concrete structure, a device for estimating a radar invisible region of a reinforced concrete structure according to this embodiment calculates a specific dielectric constant specific to the concrete on the basis of the waveform information whose vertex is visible so as to estimate a shape of the hyperbola waveform of the waveform part whose vertex is invisible.

The device for estimating the radar invisible region of the reinforced concrete structure according to this embodiment can estimate not only a horizontal position but also a depth of a reinforcing bar in the vicinity of a wall when a hyperbola waveform is acquired by radar scanning on a floor or the like, which is a reinforced concrete structure.

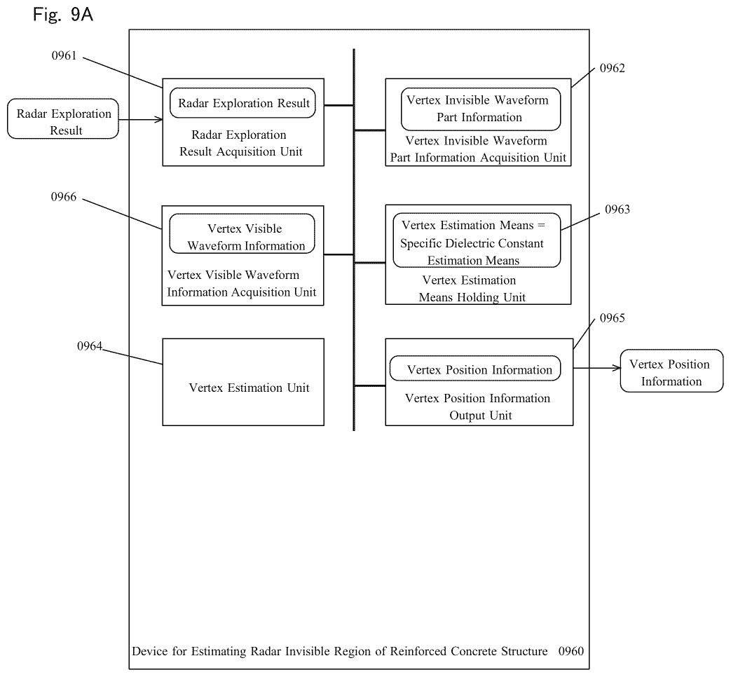

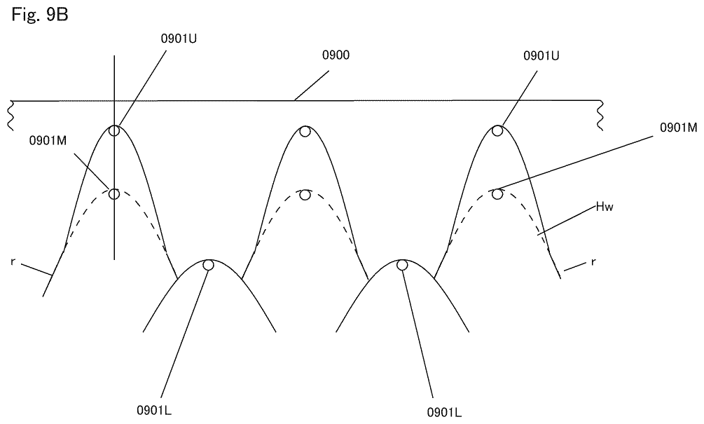

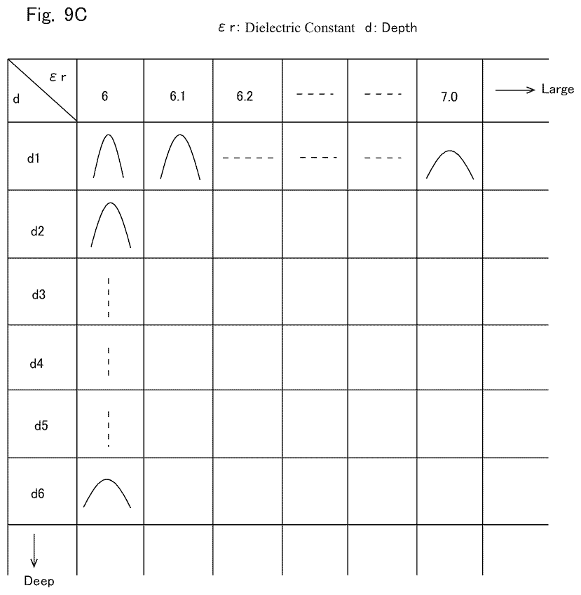

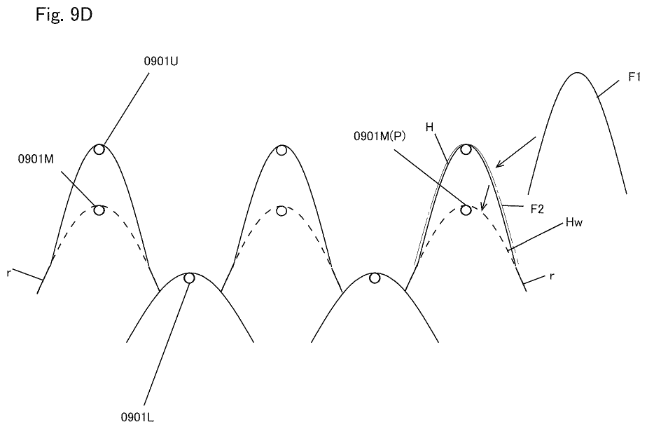

In addition, as will be described in detail in the following paragraph 0092 and FIG. 9B, the device has an effect that, when a plurality of layers of reinforcing bars are buried in the up-down direction in the floor, which is a reinforced concrete structure and the reinforcing bars are densely arranged in some spots (so-called dense arrangement), the horizontal position and the depth of the reinforcing bars in a lower layer can be estimated even if the hyperbola waveform of the reinforcing bars in a lower layer acquired by the radar scanning is hidden by the reinforcing bar on an upper layer side and thereby becomes a waveform in which the vertex is invisible and only a skirt part is visible.

### Embodiment 3: Device for Estimating Radar Invisible Region of Reinforced Concrete Structure, Configuration, Corresponding Mainly to Claims 5, 11, and 17

### Embodiment 3: Configuration, Functional Block, Entire

As shown in FIG. 9A, a device 0960 for estimating a radar invisible region of a reinforced concrete structure according to this embodiment has a radar exploration result acquisition unit 0961, a vertex invisible waveform part information acquisition unit 0962, a vertex visible waveform information acquisition unit 0966, a vertex estimation means holding unit 0963, a vertex estimation unit 0964, and a vertex position information output unit 0965.

The device 0960 for estimating the radar invisible region of the reinforced concrete structure has features as compared with the estimating devices for the radar invisible region of the reinforced concrete structure according to the above-described embodiments that: the waveform information of a waveform part whose vertex is invisible, acquired by the vertex invisible waveform part information acquisition unit 0962, is information derived from so-called dense arrangement of the reinforcing bars in the reinforced concrete structure; and vertex estimation means provided in the vertex estimation means holding unit 0963 includes specific dielectric constant estimation means.

### Embodiment 3: Device for Estimating Radar Invisible Region of Reinforced Concrete Structure, Configuration, Corresponding Mainly to Claims 5, 11, and 17

### Embodiment 3: Configuration, Functional Block, Vertex Invisible Waveform Part Information Acquisition Unit

### <Specific Example of Waveform Information of Waveform Part Whose Vertex is Invisible>

The waveform information of the waveform part whose vertex is invisible acquired in the “vertex invisible waveform part information acquisition unit 0962” is information derived from the so-called dense arrangement of the reinforcing bars in the reinforced concrete structure.

### <Dense Arrangement of Reinforcing Bars>

The “dense arrangement of the reinforcing bars” refers to the arrangement as shown in FIG. 9B, for example, in which: reinforcing bars 0901 are buried in three layers, that is, upper, middle, and lower layers, in a floor 0900, which is a reinforced concrete structure; upper-layer reinforcing bars 0901U and middle-layer reinforcing bars 0901M are in a so-called W-shaped reinforcing bar state (a state in which all the reinforcing bars is arranged with the same horizontal position); and the middle-layer reinforcing bars 0901M and lower-layer reinforcing bars 0901L are in a so-called staggered reinforcing bar state (state in which the reinforcing bars are arranged in a staggered manner to each other).

In this case, the middle-layer reinforcing bar 0901M targeted for acquisition by the radar scanning is hidden by the upper-layer reinforcing bar 0901U so that a hyperbola waveform of the middle-layer reinforcing bar 0901M becomes a partial waveform of the hyperbola waveform Hw in which the skirt r (solid-line part) is visible but the vertex (broken-line part) is invisible.

In this embodiment, the “vertex invisible waveform part information acquisition unit 0962” acquires the waveform information of the skirt r (waveform part) of the hyperbola waveform whose vertex is invisible on the basis of the middle-layer reinforcing bar 0901M.

### Embodiment 3: Device for Estimating Radar Invisible Region of Reinforced Concrete Structure, Configuration, Corresponding Mainly to Claims 5, 11, and 17

### Embodiment 3: Configuration, Functional Block, Vertex Estimation Means Holding Unit

In this embodiment, the specific dielectric constant estimation means included in the vertex estimation means held by the “vertex estimation means holding unit 0963” calculates the specific dielectric constant of the concrete on the basis of the hyperbola waveform, which is the vertex visible waveform information acquired by the vertex visible waveform information acquisition unit 0966.

### <Specific Dielectric Constant and Depth of Reinforcing Bar>

A depth D(m) of the reinforcing bar is acquired by multiplying a half (for one way) of time T(s) from when the electromagnetic wave is radiated from the transmission antenna to when it is reflected by the reinforcing bar and received by the receiving antenna by a velocity V (m/s) of the electromagnetic wave passing through the concrete, as expressed by Equation 1:

$$D = ( 1 / 2 ) \times T \times V \qquad Equation 1$$

Here, the velocity of the electromagnetic wave in the air (vacuum) is 3×10⁸ (m/s). Then, the velocity V of the electromagnetic wave passing through the concrete can be determined by using the specific dielectric constant specific to the concrete as a medium, as expressed by Equation 2:

$$V = ( 3 \times {10}^{8} ) / {( {ε}_{r} )}^{1 / 2} \qquad Equation 2$$

Thus, the depth D(m) of the reinforcing bar can be expressed by Equation 3 derived from the Equation 1 and the Equation 2:

$$D = ( 1 / 2 ) \times \{ ( 3 \times {10}^{8} ) / {( {ε}_{r} )}^{1 / 2} \} \times T \qquad Equation 3$$

The specific dielectric constant Er specific to the concrete is 6 to 8 in a standard state. Since there is the range described above in the specific dielectric constant εᵣ specific to the concrete, an accurate specific dielectric constant εᵣ of the concrete needs to be acquired in order to acquire a more accurate depth D of the reinforcing bar, that is, a hyperbola waveform, which is more accurate vertex visible waveform information of the reinforcing bar.

In this embodiment, as shown in FIG. 9C, the specific dielectric constant estimation means included in the vertex estimation means held by the vertex estimation means holding unit 0963 makes a plurality of the hyperbola figures into a database on the basis of the magnitude (6 to 8) of the specific dielectric constant εᵣ of the concrete and the buried depth d of the reinforcing bar so as to acquire the accurate specific dielectric constant of the concrete and the buried depth of the reinforcing bar by using these plurality of hyperbola figures.

In other words, from among the plurality of reinforcing bars buried in the floor, which is a reinforced concrete structure, two reinforcing bars for which vertexes of hyperbola waveforms acquired by the radar scanning are visible and the positions of the vertexes are different from each other, i.e., the upper-layer reinforcing bar 0901U and the lower-layer reinforcing bar 0901L in FIG. 9B for example, are selected. Then, on the display in the device for estimating the radar invisible region, which is a computer such as a laptop personal computer, or on the radar screen of the electromagnetic wave radar, the plurality of hyperbola figures made into the database are selectively superimposed on at least one of the two hyperbola waveforms with respect to the reinforcing bars 0901U and 0901L, and a numeral value displayed on the radar screen at a point of time when the hyperbola waveform and the hyperbola figure are overlapped is calculated as the specific dielectric constant εᵣ of the concrete of the floor, which is the reinforced concrete structure.

It is to be noted that it may be so configured that, in the specific dielectric constant estimation means included in the vertex estimation means held by the vertex estimation means holding unit 0963, the specific dielectric constant of the concrete is calculated by using the equation y=f(x) of the curve derived from the hyperbola waveform, which is the vertex visible waveform information acquired by the vertex visible waveform information acquisition unit 0966.

In other words, in FIG. 9B for example, the upper-layer reinforcing bar 0901U and the lower-layer reinforcing bar 0901L may be selected so as to calculate the specific dielectric constant of the concrete by using the following Equations 4 and 5 derived from the two hyperbola waveforms related to these reinforcing bars 0901U and 0901L:

$$y = {f}_{1} ( x , D 1 , {ε}_{r} ) \qquad Equation 4$$

$$y = {f}_{2} ( x , D 2 , {ε}_{r} ) \qquad Equation 5$$

Here, “D1” and “D2” in the Equations 4 and 5 are apparent depths of the reinforcing bars 0901U and 0901L, respectively, acquired by the aforementioned Equation 3 by provisionally setting the specific dielectric constant εᵣ specific to the concrete to a standard value of 6. Moreover, “x” in the Equations 4 and 5 is a distance from the scanning start point measured by the distance meter and is a known value.

In order to calculate a more accurate specific dielectric constant, a diameter of the reinforcing bar needs to be considered. However, consideration on the diameter is omitted in this specification in order to avoid complication.

### Embodiment 3: Configuration, Functional Block, Vertex Estimation Unit

In this embodiment, the “vertex estimation unit 0964” functions to estimate the vertex position of the vertex invisible waveform part on the basis of: the vertex visible waveform information acquired by the vertex visible waveform information acquisition unit 0966; the vertex invisible waveform part information acquired by the vertex invisible waveform part information acquisition unit 0962; and the specific dielectric constant estimation means, which is the vertex estimation means of the vertex estimation means holding unit 0963.

Specifically, as shown in FIG. 9D, a hyperbola figure F1 is displayed as a template on the display in the device for estimating the radar invisible region, which is a computer such as a laptop personal computer, or on the radar screen of the electromagnetic wave radar, on which the hyperbola waveforms of the upper-layer reinforcing bar 0901U and the lower-layer reinforcing bar 0901L, which are the vertex visible waveform information acquired by the vertex visible waveform information acquisition unit 0966, and a hyperbola waveform of the middle-layer reinforcing bar 0901M in which only the skirt is visible but the vertex is invisible, which is the vertex invisible waveform part information acquired by the vertex invisible waveform part information acquisition unit 0962, have been displayed. This hyperbola figure F1 is a figure created as the accurate specific dielectric constant εᵣ calculated by the aforementioned specific dielectric constant estimation means, and as explained in the “Characteristics of Hyperbola Waveform” in the aforementioned paragraph 0059, the vertex of the mountain shape of the hyperbola figure changes in accordance with a change in the depth.

Then, on the radar screen, for example, the hyperbola figure F1 is moved in a direction of an arrow and fitted to the hyperbola waveform H of the upper-layer reinforcing bar 0901U so as to acquire a hyperbola figure F2 as a one dot chain line that best fits the hyperbola waveform of the upper-layer reinforcing bar 0901U. Thereafter, this hyperbola figure F2 is moved in a direction of the hyperbola waveform Hw of the middle-layer reinforcing bar 0901M which is the vertex invisible waveform part information and in which only the skirt r is visible and the vertex is invisible, and is curve-fitted to the hyperbola waveform Hw whose vertex is invisible so as to estimate the position of the vertex P (middle-layer reinforcing bar 0901M) including the reinforcing-bar depth of the vertex invisible waveform part.

### Embodiment 3: Device for Estimating Radar Invisible Region of Reinforced Concrete Structure, Hardware Configuration, Corresponding Mainly to Claims 5, 11, and 17

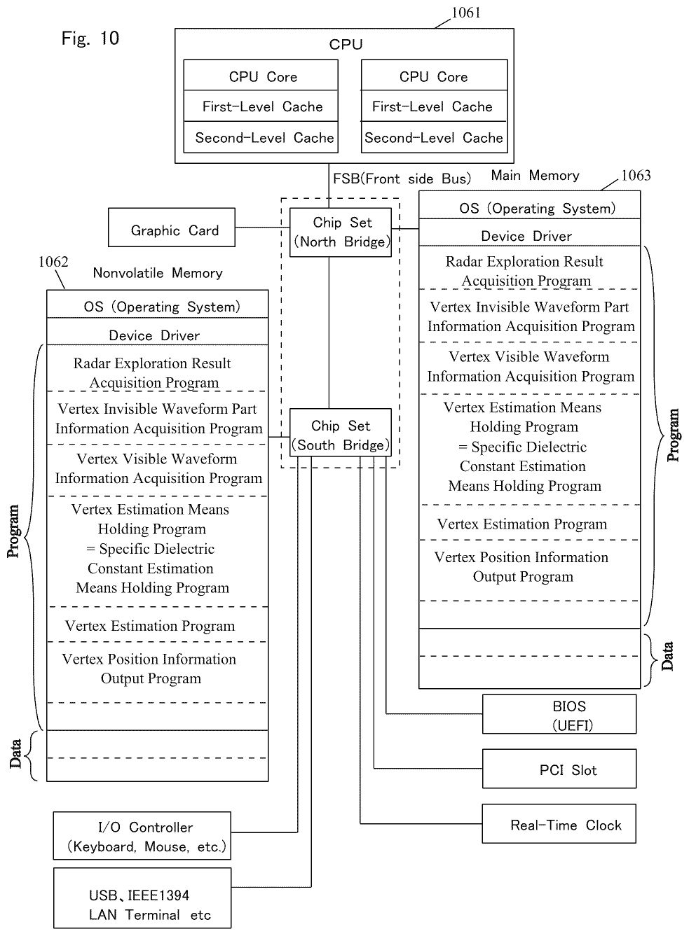

As shown in FIG. 10, a device 1060 for estimating a radar invisible region of a reinforced concrete structure according to this embodiment consists of a CPU 1061, a nonvolatile memory 1062 such as an HDD or a ROM, a main memory 1063 such as a D-RAM, and an interface. The nonvolatile memory 1062 stores a radar exploration result acquisition program, a vertex invisible waveform part information acquisition program, a vertex visible waveform information acquisition program, a vertex estimation means holding program, a vertex estimation program, and a vertex position information output program, and the plural estimation means as the vertex estimation means is used in the vertex estimation means holding program. The data is information on a current signal and a phase angle, and these programs and data are read into a holding area of the main memory 1063 and executed in an operation area. Moreover, the interface includes a specific low-power radio and the like.

### Embodiment 3: Device for Estimating Radar Invisible Region of Reinforced Concrete Structure, Flow of Processing, Corresponding Mainly to Claims 5, 11, and 17

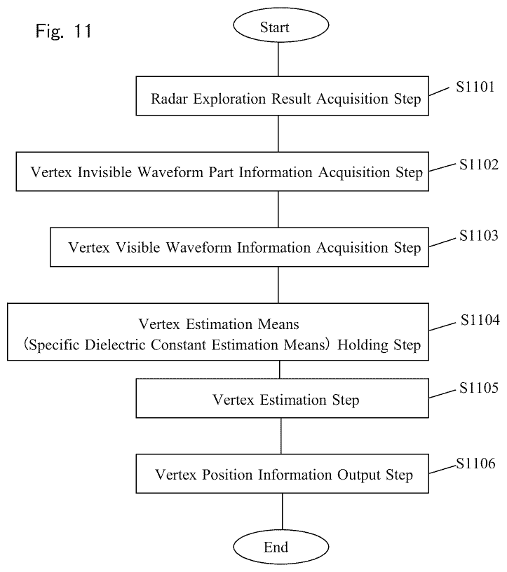

In the device for estimating the radar invisible region of the reinforced concrete structure according to this embodiment, as shown in FIG. 11, a radar exploration result acquisition step S1101 is executed for acquiring a radar exploration result of the reinforced concrete structure.

Subsequently, a vertex invisible waveform part information acquisition step S1102 is executed for acquiring the waveform information of the waveform part of the hyperbola waveform whose vertex is invisible from the acquired radar exploration result. Then, the vertex visible waveform information acquisition step S1103 is executed for acquiring the waveform information of the vertex visible waveform.

Subsequent to the above, a vertex estimation means holding step S1104 is executed for holding the plural estimation means as estimation means for estimating the vertex position of the vertex invisible waveform part on the basis of the vertex visible waveform information and the vertex invisible waveform part information.

Then, a vertex estimation step S1105 is executed for estimating the vertex position of the vertex invisible waveform part on the basis of the vertex visible waveform information, the vertex invisible waveform part information, and the plural estimation means as the vertex estimation means. Thereafter, the vertex position information output step S1106 is executed for outputting the estimated vertex position information.

### Embodiment 3: Device for Estimating Radar Invisible Region of Reinforced Concrete Structure, Effects, Corresponding Mainly to Claims 5, 11, and 17

The device for estimating the radar invisible region of the reinforced concrete structure according to this embodiment has an effect that not only a horizontal position but also a depth of a reinforcing bar in the vicinity of a wall can be estimated when a hyperbola waveform is acquired by radar scanning on a floor or the like, which is a reinforced concrete structure, as well as an effect that, when a plurality of layers of reinforcing bars are buried in the up-down direction in the floor, which is a reinforced concrete structure and the reinforcing bars are densely arranged in some spots (so-called dense arrangement), the horizontal position and the depth of the reinforcing bars in a lower layer can be estimated even if the hyperbola waveform of the reinforcing bars in a lower layer acquired by the radar scanning is hidden by the reinforcing bar on an upper layer side and thereby becomes a waveform in which the vertex is invisible and only a skirt part is visible.

1. A device for estimating a radar invisible region of a reinforced concrete structure comprising:
a radar exploration result acquisition unit for the reinforced concrete structure;
a vertex invisible waveform part information acquisition unit which acquires waveform information of a waveform part of a hyperbola waveform in which a vertex is invisible from the acquired radar exploration result;
a vertex estimation means holding unit which holds vertex estimation means for estimating a vertex position of the vertex invisible waveform part on the basis of the vertex invisible waveform part information; and
a vertex estimation unit which estimates a vertex position of the vertex invisible waveform part on the basis of the vertex invisible waveform part information and the vertex estimation means.

2. The device for estimating the radar invisible region of the reinforced concrete structure according to claim 1 comprising a vertex visible waveform information acquisition unit which acquires waveform information of a vertex visible waveform, wherein:
the vertex estimation means of the vertex estimation means holding unit is further configured to estimate the vertex position of the vertex invisible waveform part also on the basis of the vertex visible waveform information; and
the vertex estimation unit estimates the vertex position of the vertex invisible waveform part on the basis of the vertex visible waveform information, the vertex invisible waveform part information, and the vertex estimation means.

3. The device for estimating the radar invisible region of the reinforced concrete structure according to claim 1 further comprising a vertex position information output unit which outputs the estimated vertex position information.

4. The device for estimating the radar invisible region of the reinforced concrete structure according to claim 2, wherein the vertex estimation means is plural estimation means which estimates a shape of a hyperbola curve of the vertex invisible waveform part on the basis of a plurality of pieces of the vertex visible waveform information.

5. The device for estimating the radar invisible region of the reinforced concrete structure according to claim 2, wherein the vertex estimation means includes specific dielectric constant estimation means which estimates the shape of the hyperbola curve of the vertex invisible waveform part by calculating a specific dielectric constant of a concrete on the basis of the vertex visible waveform information.

6. The device for estimating the radar invisible region of the reinforced concrete structure according to claim 1, wherein the vertex invisible waveform part is a vertex invisible waveform part which is present in a difficult part where a radar exploration by a radar exploration device is difficult due to a shape of the concrete structure.

7. A method of operating a device, which is a computer, for estimating a radar invisible region of a reinforced concrete structure, comprising:
a radar exploration result acquisition step for a reinforced concrete structure;
a vertex invisible waveform part information acquisition step for acquiring waveform information of a waveform part of a hyperbola waveform in which a vertex is invisible in the acquired radar exploration result;
a vertex estimation means holding step for holding the vertex estimation means for estimating a vertex position of the vertex invisible waveform part on the basis of the vertex invisible waveform part information; and
a vertex estimation step for estimating the vertex position of the vertex invisible waveform part on the basis of the vertex invisible waveform part information and the vertex estimation means.

8. The method of operating the device, which is the computer, for estimating the radar invisible region of the reinforced concrete structure according to claim 7, comprising a vertex visible waveform information acquisition step for acquiring waveform information of a vertex visible waveform, wherein:
the vertex estimation means of the vertex estimation means holding step is further configured to estimate the vertex position of the vertex invisible waveform part also on the basis of the vertex visible waveform information; and
the vertex estimation step estimates the vertex position of the vertex invisible waveform part on the basis of the vertex visible waveform information, the vertex invisible waveform part information, and the vertex estimation means.

9. The method of operating the device, which is the computer, for estimating the radar invisible region of the reinforced concrete structure according to claim 7, comprising a vertex position information output step for outputting the estimated vertex position information.

10. The method of operating the device, which is the computer, for estimating the radar invisible region of the reinforced concrete structure according to claim 8, wherein the vertex estimation means is plural estimation means which estimates a shape of a hyperbola curve of the vertex invisible waveform part on the basis of a plurality of pieces of the vertex visible waveform information.

11. The method of operating the device, which is the computer, for estimating the radar invisible region of the reinforced concrete structure according to claim 8, wherein the vertex estimation means includes specific dielectric constant estimation means which estimates the shape of the hyperbola curve of the vertex invisible waveform part by calculating a specific dielectric constant of a concrete on the basis of the vertex visible waveform information.

12. The method of operating the device, which is the computer, for estimating the radar invisible region of the reinforced concrete structure according to claim 7, wherein the vertex invisible waveform part is a vertex invisible waveform part which is present in a difficult part where a radar exploration by a radar exploration device is difficult due to a shape of the concrete structure.

13. A method of estimating a radar invisible region of a reinforced concrete structure which can be read and executed by a computer, comprising:
a radar exploration result acquisition program of a reinforced concrete structure;
a vertex invisible waveform part information acquisition program for acquiring waveform information of a waveform part of a hyperbola waveform in which a vertex is invisible in the acquired radar exploration result;
a vertex estimation means holding program for holding the vertex estimation means for estimating a vertex position of the vertex invisible waveform part on the basis of the vertex invisible waveform part information; and
a vertex estimation program for estimating the vertex position of the vertex invisible waveform part on the basis of the vertex invisible waveform part information and the vertex estimation means.

14. The method of estimating the radar invisible region of the reinforced concrete structure which can be read and executed by the computer according to claim 13, comprising a vertex visible waveform information acquisition program for acquiring waveform information of a vertex visible waveform, wherein:
the vertex estimation means of the vertex estimation means holding program is further configured to estimate the vertex position of the vertex invisible waveform part also on the basis of the vertex visible waveform information; and
the vertex estimation program estimates the vertex position of the vertex invisible waveform part on the basis of the vertex visible waveform information, the vertex invisible waveform part information, and the vertex estimation means.

15. The method of estimating the radar invisible region of the reinforced concrete structure which can be read and executed by the computer according to claim 13, further comprising a vertex position information output program for outputting the estimated vertex position information.

16. The method of estimating the radar invisible region of the reinforced concrete structure which can be read and executed by the computer according to claim 13, wherein the vertex estimation means is plural estimation means which estimates a shape of a hyperbola curve of the vertex invisible waveform part on the basis of a plurality of pieces of the vertex visible waveform information.

17. The method of estimating the radar invisible region of the reinforced concrete structure which can be read and executed by the computer according to claim 13, wherein the vertex estimation means includes specific dielectric constant estimation means which estimates the shape of the hyperbola curve of the vertex invisible waveform part by calculating a specific dielectric constant of a concrete on the basis of the vertex visible waveform information.

18. The method of estimating the radar invisible region of the reinforced concrete structure which can be read and executed by the computer according to claim 13, wherein the vertex invisible waveform part is a vertex invisible waveform part which is present in a difficult part where a radar exploration by a radar exploration device is difficult due to a shape of the concrete structure.
